(19) United States
(12) Patent Application Publication
(10) Pub. No.: US 2021/0282705 A1
(43) Pub. Date: Sep. 16, 2021

(54) SYSTEMS AND METHODS FOR MODELING SLEEP PARAMETERS FOR A SUBJECT

(71) Applicant: KONINKLIJKE PHILIPS N.V., Eindhoven (NL)

(72) Inventors: Gary Nelson GARCIA MOLINA, Madison, WI (US); Benjamin Irwin SHELLY, Pittsburgh, PA (US); Anthony BREWER, Pittsburgh, PA (US); Matthew HOGAN, Pittsburgh, PA (US)

(21) Appl. No.: 17/201,026

(22) Filed: Mar. 15, 2021

Related U.S. Application Data
(60) Provisional application No. 62/990,110, filed on Mar. 16, 2020.
(60) Provisional application No. 63/046,391, filed on Jun. 30, 2020.

(51) Int. Cl. A61B 5/00; A61B 5/0205; A61B 5/021; A61B 5/11; G16H 50/50

(52) U.S. Cl. CPC A61B 5/4809; A61B 5/0205; G16H 50/50; A61B 5/1114; A61B 5/021

(57) ABSTRACT

The present disclosure pertains to systems and methods for modeling at least a sleep parameter for a subject. In some embodiments, a method for predicting at least a sleep parameter for a subject comprises: obtaining sleep and/or wake information related to a subject for a given period of time; estimating bedtime and/or wakeup time of the subject using a sleep model and the sleep and/or wake information; predicting at least a sleep parameter of the subject for an upcoming time interval based upon the sleep model and the estimated bedtime and/or wakeup time; obtaining physiological information of the subject; and adjusting the predicted sleep parameter for the upcoming time interval based upon the sleep model and the physiological information.

### CROSS-REFERENCE TO PRIOR APPLICATIONS

This application claims the benefit of U.S. Provisional Application No. 62/990,110, filed on 16 Mar. 2020 and U.S. Provisional Application No. 63/046,391, filed on 30 Jun. 2020. These applications are hereby incorporated by reference herein.

### BACKGROUND

### 1. Field

The present disclosure pertains to systems and methods for modeling at least a sleep parameter for a subject.

### 2. Description of the Related Art

A general premise of sleep/wake regulation is that the longer one is awake, the shorter time it takes to fall asleep. However, not all “wake” periods accumulate sleep-need equivalently. Considering timing of sleep and wake alone are not sufficient to accurately model the dynamics of sleep-need. Numerous factors play a role in sleep/wake regulation and these cannot be all included in a model. The present disclosure overcomes these deficiencies.

### SUMMARY

Accordingly, one or more aspects of the present disclosure relates to a system for modeling at least a sleep parameter for a subject. The system comprises a non-transitory computer readable medium having instructions thereon, the instructions when executed by a computer causing the computer to: obtain sleep and/or wake information related to a subject for a given period of time; estimate bedtime and/or wakeup time of the subject using a sleep model and the sleep and/or wake information; predict at least a sleep parameter of the subject for an upcoming time interval based upon the sleep model and the estimated bedtime and/or wakeup time; obtain physiological information of the subject; and adjust the predicted sleep parameter for the upcoming time interval based upon the sleep model and the physiological information.

Another aspect of the present disclosure relates to a method for modeling at least a sleep parameter for a subject. The method comprises: obtain sleep and/or wake information related to a subject for a given period of time; estimate bedtime and/or wakeup time of the subject using a sleep model and the sleep and/or wake information; predict at least a sleep parameter of the subject for an upcoming time interval based upon the sleep model and the estimated bedtime and/or wakeup time; obtain physiological information of the subject; and adjust the predicted sleep parameter for the upcoming time interval based upon the sleep model and the physiological information.

Still another aspect of the present disclosure relates to a system for modeling at least a sleep parameter for a subject. The system comprises one or more input devices configured to generate output signals indicating one or more physiological information related to a subject, and one or more physical processors operatively connected with the one or more input devices, the one or more physical processors configured by machine-readable instructions to: obtain sleep and/or wake information related to a subject for a given period of time; estimate bedtime and/or wakeup time of the subject using a sleep model and the sleep and/or wake information; predict at least a sleep parameter of the subject for an upcoming time interval based upon the sleep model and the estimated bedtime and/or wakeup time; obtain physiological information of the subject; and adjust the predicted sleep parameter for the upcoming time interval based upon the sleep model and the physiological information.

Still another aspect of the present disclosure relates to a system for modeling at least a sleep parameter for a subject, the system comprising: obtaining means for obtaining sleep and/or wake information related to a subject for a given period of time; estimating means for estimating bedtime and/or wakeup time of the subject using a sleep model and the sleep and/or wake information; predicting means for predicting at least a sleep parameter of the subject for an upcoming time interval based upon the sleep model and the estimated bedtime and/or wakeup time; obtaining means for obtaining physiological information of the subject; and adjusting means for adjusting the predicted sleep parameter for the upcoming time interval based upon the sleep model and the physiological information.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system for modeling at least a sleep parameter for a subject, in accordance with one or more embodiments;

FIG. 2 illustrates a sleep model, in accordance with one or more embodiments;

FIG. 3 illustrates an example of sleep latency, in accordance with one or more embodiments;

FIG. 4 illustrates an example operations of the system, in accordance with one or more embodiments;

FIG. 5 illustrates an example correlation between eye movement and sleepiness level, in accordance with one or more embodiments;

FIG. 6a illustrates an example of an adjusted sleep model, in accordance with one or more embodiments;

FIG. 6b illustrates an example of an adjusted sleep model, in accordance with one or more embodiments;

FIG. 7 illustrates an example of a correlation between physiological parameters and a sleepiness scale, in accordance with one or more embodiments;

FIG. 8 illustrates an example of a correlation between a subjective sleepiness scale and a sleepiness scale, in accordance with one or more embodiments;

FIG. 9 illustrates an example of a correlation between a performance on a sleepiness test and a sleepiness scale, in accordance with one or more embodiments; and

FIG. 10 illustrates a method for modeling at least a sleep parameter for a subject, in accordance with one or more embodiments.

### DETAILED DESCRIPTION OF EXEMPLARY EMBODIMENTS

As used herein, the singular form of “a,” “an,” and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein such as, for example and without limitation, top, bottom, left, right, upper, lower, front, back, and derivatives thereof, relate to the orientation of the elements shown in the drawings and are not limiting upon the claims unless expressly recited therein.

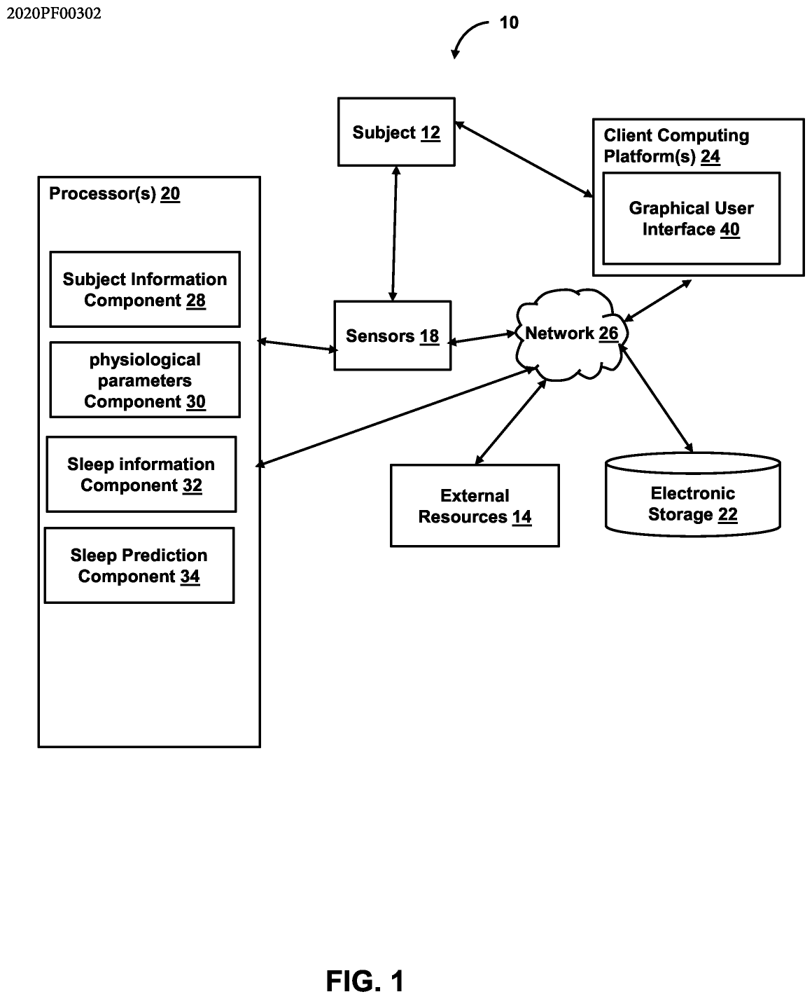

FIG. 1 is a schematic illustration of a system 10 for modeling at least a sleep parameter for a subject, in accordance with one or more embodiments. System 10 may be configured to provide subjects with predictive information on future sleep quality based on the prediction of a base model refined by information extracted from daytime physiology or behavior monitoring. In some embodiments, predictions of the model may be corrected when new data becomes available. This approach may be advantageous because it does not attempt to incorporate multiple variables in the model which can unnecessarily increase complexity and compromise its usefulness. The predictive nature of the information on future sleep quality may be relevant to users because it can guide their behavior. For example, users may know which activities promote higher sleep quality.

In some embodiments, system 10 comprises one or more of sensor(s) 18, a processor 20, external resources 14, electronic storage 22, client computing platform(s) 24, a network 26, and/or other components. In FIG. 1, sensor(s) 18, processor 20, electronic storage 22, and client computing platform(s) 24 are shown as separate entities. In some embodiments, some or all of the components of system 10 and/or other components may be grouped into one or more singular user devices (e.g., a wearable device, a mobile phone, a sensing device, a medical device, a computing device, or other user devices). In some embodiments, the user device may include a housing, one or more sensors (e.g., sensors 18), processors (e.g., processors 20), storage (e.g., electronic storage 22), user interface (e.g., client computing platform(s) 24), and/or other components. In some embodiments, the one or more sensors, processors, storage, user interface, and other components of system 10 (e.g., the user device) may all be housed within the housing. In some embodiments, some of the components of system 10 may be housed outside the housing. In some embodiments, such sensors, processors, and other components of the user device may communicate with one another via wired or wireless connections. In some embodiments, a user device may be configured to perform certain operations of system 10. In some embodiments, one or more such operations may be performed by one or more other components (e.g., one or more servers, client devices, etc.). As an example, such other components (e.g., one or more servers, client devices, etc.) may include one or more processor components that are the same as or similar to subsystems components 28-34.

Sensor(s) 18 is configured to generate output signals conveying information related to one or more physiological information related to subject 12. In some embodiments, the physiological information of the subject may include one or more of heart rate, heart rate variability, microvascular blood volume, electrical function of the heart, brain activity, eye movement, physical activity, sleep/wake status, sleep duration, wake duration, sleep/wake onset, and/or other physiological information. In some embodiments, the one or more sensor(s) may include one or more of a heart rate sensor, an electrocardiogram (ECG), a photoplethysmograph (PPG), an electroencephalogram (EEG), and electrooculography, and/or other sensors. In some embodiments, sensor(s) 18 may include a pulse oximeter, a movement sensor, an accelerometer, a blood pressure sensor, an actimetry sensor, a camera, a breathing sensor, and/or other sensors configured for monitoring the subject state. Although sensor(s) 18 is illustrated at a single location near subject 12, this is not intended to be limiting. In some embodiments, sensor(s) 18 may include sensors disposed in a plurality of locations, (e.g., sensor disposed on (or near) the chest, limb, head, ear, eye, and/or other body parts of subject 12. In some embodiments, sensor(s) 18 may include sensors coupled (in a removable manner) with clothing of subject 12. In some embodiments, sensor(s) 18 may include sensors disposed in a computing device (e.g., a mobile phone, a computer, etc.), and/or disposed in a medical device used by the user. In some embodiments, sensor(s) 18 may include sensors that are positioned to point at subject 12 (e.g., a camera).

In some embodiments, sensor(s) 18 may be included in a wearable device. The wearable device may be any device that is worn, or that is in full or partial contact with any body part of the subject. In some embodiments, the wearable device may be in the form of a wristband, an activity monitor, a smart watch, a headband, ear plugs, etc. In some embodiments, the wearable device may be configured to generate output signals conveying information related to heart rate, heart rate variability, microvascular blood volume, electrical function of the heart, brain activity, eye movement, physical activity, sleep/wake status, sleep duration, wake duration, sleep/wake onset, and/or other physiological information, and/or other physiological parameters. The output signals may be transmitted to a computing device (within or outside of the wearable device) wirelessly and/or via wires. In some embodiments, some or all components of system 10 may be included in the wearable device.

Processor 20 is configured to provide information processing capabilities in system 10. As such, processor 20 may include one or more of a digital processor, an analog processor, and a digital circuit designed to process information, an analog circuit designed to process information, a state machine, and/or other mechanisms for electronically processing information. Although processor 20 is shown in FIG. 1 as a single entity, this is for illustrative purposes only. In some embodiments, processor 20 may include a plurality of processing units. These processing units may be physically located within the same device (e.g., a server), or processor 20 may represent processing functionality of a plurality of devices operating in coordination (e.g., one or more servers, one or more computing platforms 24 associated with users, a medical device, sensor(s) 18, a piece of a hospital equipment, devices that are part of external resources 14, electronic storage 22, and/or other devices.)

As shown in FIG. 1, processor 20 is configured to execute one or more computer program components. The one or more computer program components may comprise one or more of a subject information component 28, a physiological parameters component 30, a sleep information component 32, a sleep prediction component 34, and/or other components. Processor 20 may be configured to execute components 28, 30, 32, 34, and/or other components by software; hardware; firmware; some combination of software, hardware, and/or firmware; and/or other mechanisms for configuring processing capabilities on processor 20.

It should be appreciated that although components 28, 30, 32, and 34 are illustrated in FIG. 1 as being co-located within a single processing unit, in embodiments in which processor 20 comprises multiple processing units, one or more of components 28, 30, 32, 34, and/or other components may be located remotely from the other components. The description of the functionality provided by the different components 28, 30, 32, 34 and/or other components described below is for illustrative purposes, and is not intended to be limiting, as any of components 28, 30, 32, and 34 may provide more or less functionality than is described. For example, one or more of components 28, 30, 32, and 34 may be eliminated, and some or all of its functionality may be provided by other components 28, 30, 32, and 34. As another example, processor 20 may be configured to execute one or more additional components that may perform some or all of the functionality attributed below to one of components 28, 30, 32, and/or 34.

Subject information component 28, in some embodiments, is configured to obtain information related to subject 12. In some embodiments, information related to subject 12 may include biographical information. For example, biographical information may include demographic information (e.g., gender, ethnicity, age, etc.), vital sign information (e.g., height, weight, BMI, blood pressure, pulse, temperature, respiration, etc.), medical/health condition information (e.g., a disease type, severity of the disease, stage of the disease, categorization of the disease, symptoms, behaviors, readmission, relapse, etc.), treatment history information (e.g., type of treatments, length of treatments, current and past medications, etc.), and/or other information.

In some embodiments, subject information component 28 may be configured to determine (and/or obtain) information related to other subjects. For example, subjects with similar sleep and/or wake information, demographic information, vital sign information, medical/health condition information, treatment history information, similar desired outcome (e.g., from sensory simulation), and/or other similarities with subject 12. It should be noted that the subject information described above is not intended to be limiting. A large number of information related to subjects may exist and may be used with system 10 in accordance with some embodiments. For example, users may choose to customize system 10 and include any type of subject data they deem relevant.

In some embodiments, subject information component 28 may be configured to obtain/extract information from one or more databases (e.g., electronic storage 22 shown in FIG. 1). In some embodiments, different databases may contain different information about subject 12 and/or about other subjects (e.g. similar to subject 12). In some embodiments, some databases may be associated with specific subject information (e.g., sleep and/or wake information, a medical condition, a demographic characteristic, a treatment, a therapy, a medical device used, a vital sign information, etc.) In some embodiments, subject information component 28 may be configured to obtain/extract the subject information from external resources 14 (e.g., one or more external databases included in external resources 14), electronic storage 22 included in system 10, one or more medical devices, and/or other sources of information.

Physiological parameters component 30 may be configured to determine (and/or obtain) one or more physiological parameters related to subject 12. In some embodiments, the one or more physiological parameters may be determined based on output signals from sensor(s) 18. In some embodiments, the one or more physiological parameters may include heart rate, heart rate variability, microvascular blood volume, electrical function of the heart, brain activity, eye movement, physical activity, sleep/wake status, sleep duration, wake duration, sleep/wake onset, and/or other physiological information, alpha power, theta power, and/or other physiological parameters. In some embodiments, the one or more physiological parameters may be determined before, during, and/or after a sleep session. For example, in some embodiments, the one or more physiological parameters may be determined between the time the subject wakes and the time he goes to bed.

In some embodiments, the one or more physiological parameters related to subject 12 may include information related to one or more physical activities of the subject (e.g., standing, sitting, walking, running, exercising, relaxing, and/or other physical activities). In some cases, medical treatment of the subject may influence their sleepiness, drowsiness, sleep need, sleep quality, and/or other sleep parameters of the subject. For example, the information about the physical activity may include a type of physical activity, intensity, duration, time of the day the activity was carried out, and/or other physical activities. In some cases, the information about the physical activity may be that the user did not carry out any physical activity.

In some embodiments, the one or more physiological parameters related to subject 12 may include information related to one or more dietary information of the subject (e.g., food and/or drink). In some cases, diet of the subject may influence their sleepiness, drowsiness, sleep need, sleep quality, and/or other sleep parameters of the subject (e.g., caffeine, alcohol, carbohydrates, fatty foods, spicy foods, and/or other foods). In some embodiments, the dietary information may include one or more types of drinks and/or foods, amount of drinks and/or foods, time of day the drinks and/or foods were consumed, and/or other dietary information.

In some embodiments, the one or more physiological parameters related to subject 12 may include information related to one or more medical treatment (e.g., drug, medical intervention, therapy, and/or other medical treatment). In some cases, medical treatment of the subject may influence their sleepiness, drowsiness, sleep need, sleep quality, and/or other sleep parameters of the subject. For example, in some embodiments, the medical treatment information may include one or more types of medical treatment, dosage, time of day the medical treatment is taken, and/or other medical treatment information.

In some embodiments, the one or more physiological parameters related to subject 12 may include psychological information related to the subject (e.g., stress, anxiety, and/or other psychological information). In some cases, psychological information of the subject may influence their sleepiness, drowsiness, sleep need, sleep quality, and/or other sleep parameters of the subject. For example, the psychological information may include a type of psychological information, intensity level, time of the day it occurs, and/or other psychological information of the subject.

In some embodiments, the one or more physiological parameters related to subject 12 may include information related to daytime nap information of the subject. In some cases, daytime napping may influence the subject sleep need, sleep debt, sleep/wake quality, and/or other sleep parameters of the subject. For example, in some embodiments, the daytime nap information may include one or more of the length, quality, time of the nap, and/or other daytime information.

In some embodiments, physiological parameters component 30 is configured to obtain the physiological parameters from one or more databases within or outside system 10. For example, electronic storage 22, external resources 14, and/or other databases. In some embodiments, the one or more physiological parameters may be obtained from one or more sensors outside of system 10. For example, in some embodiments, system 10 does not require sensors 18, instead it receives information from outside sensors that are related to the subject. The information may be in the form of output signals that physiological parameters component 30 uses to determine the physiological parameters, and/or the information obtained is physiological parameters that do not require additional processing from system 10. In some embodiments, this information may be sent automatically to system 10 whenever it becomes available, or system 10 may be configured to request the information (e.g., in a continuous manner, and/or on a schedule). For example, in some embodiments, the outside sensors may be independent physiological sensors, sensors included in activity devices, medical devices, mobile phones, smart wearables devices, and/or other sensors. In some embodiments, the physiological parameters are obtained via an input device (e.g., client computing platform(s) 24). The user may use the input device to provide physiological parameters to system 10. For example, the input device may be a wearable device, a smart phone, a smart watch, a computer, and/or any other device that is able to communicate with system 10.

Sleep information component 32 may be configured to obtain sleep information of the subject. For example, sleep information may include bedtime and/or wakeup time of the subject, sleep/wake status, sleep duration, wake duration, sleep/wake onset, sleep latency, sleep need, sleep debt, and/or other sleep parameters of the subject. In some embodiments, sleep information may include historical sleep information. For example, in some embodiments, sleep information component 32 may be configured to obtain sleep information over a period of time (e.g., 24h, 48h, few days, weeks, months, years, or any period of time.)

In some embodiments, sleep information component 32 is configured to obtain the sleep information from one or more databases within or outside system 10. For example, electronic storage 22, external resources 14, and/or other databases. In some embodiments, the one or more sleep information may be obtained from one or more sensors outside of system 10. For example, in some embodiments, system 10 does not require sensors 18, instead it receives information from outside sensors that are related to the subject. The information may be in the form of output signals that sleep information component 32 uses to determine the sleep information, and/or the information obtained is physiological parameters that do not require additional processing from system 10. For example, in some embodiments, the outside sensors may be independent physiological sensors, sensors included in activity devices, medical devices, mobile phones, smart wearables devices, and/or other sensors. In some embodiments, the sleep information is obtained via an input device (e.g., client computing platform(s) 24). The user may use the input device to provide sleep information to system 10. For example, the input device may be a wearable device, a smart phone, a smart watch, a computer, and/or any other device that is able to communicate with system 10.

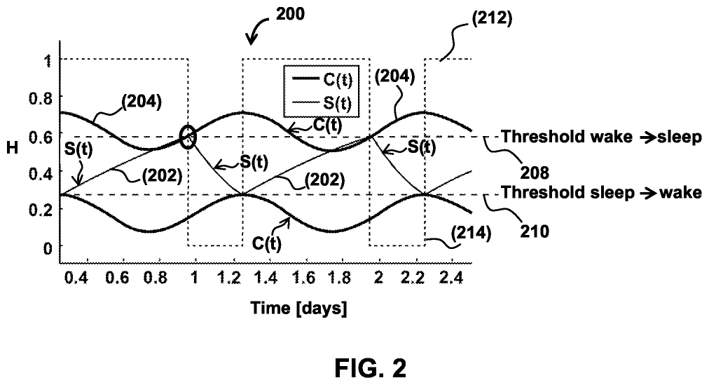

Sleep information component 32, in some embodiments, may be configured to estimate one or more sleep parameters of the subject. In some embodiments, the one or more sleep parameters of the subject may be estimated using one or more sleep models. In some embodiments, parameters of a sleep model of the one or more sleep models may be obtained using a two-process model. Generally, sleep and wake periods alternate throughout a 24-hour cycle and they are regulated by two factors: homeostatic and circadian. In some embodiments, an example sleep model of sleep/wake regulation is a two-process model where a homeostatic component “H” (see FIG. 2 described below) models the accumulation of sleep-need during wakefulness through an exponentially saturating curve and during sleep models the dissipation of sleep-need using an exponentially decaying curve. In this model, the circadian factor determines the timing of sleep and wake through two “parallel” 24-hour period sinusoidal curves (see FIG. 2 described below).

FIG. 2 illustrates a sleep model 200 in accordance with one or more embodiments. Sleep model 200 is a two-process model where a homeostatic component H models the accumulation of sleep-need during wakefulness 212 through an exponentially saturating curve (S(t))202. During sleep 214, the homeostatic component H models the dissipation of sleep-need using an exponentially decaying curve 202. In this model, the circadian factor determines the timing of sleep and wake through two “parallel” 24-hour period sinusoidal curves (C(t)) 204. A threshold wake to sleep 208 is defined by homeostatic saturating curve 202 meeting the circadian curve 204. A threshold sleep to wake 210 is defined by homeostatic decaying curve 202 meeting the circadian curve 204.

We consider that at time “t” the signal s(t) is sampled and that a relation Sₕ exists such that an estimation of H at time t: [symbol] can be obtained from s(t): [symbol] =Sₕ(s(t)). The function Sₕ depends on the specific type of signal and the specific instances thereof are detailed herein below. The homeostatic component H models the accumulation of sleep-need during wakefulness according to the following equation:

H(t)during wake:H(t)=μ+(H₀−μ)exp(t₀−t)/τw

During sleep 214, the homeostatic component H models the dissipation of sleep-need according to the following equation:

H(t)during sleep: H(t)=Hₑ exp(t₀−t)/τs

The circadian factor determines the timing of sleep and wake through two “parallel” 24-hour period sinusoidal curves (C(t)) 204 according to the following equations:

C(t): Cᵤₚ(t)=H₀⁺+0.1 sin(2πt)

C_{dwn}(t)=H₀⁻+0.1 sin(2πt)

where the sleep model parameters are the average (24-hour format) bedtime and wakeup time, the asymptotic value μ for H during wakefulness, the time constant τw which controls the rate at which sleep-need accumulates during wake, the time constant τs which controls the rate at which sleep-need dissipates during the sleep period, and the upper and lower circadian shift parameters H₀⁺ and H₀⁻ respectively. In some embodiments, the sleep model parameters may be estimated from a sequence of sleep/wake history. For example, a sleep/wake history of a few days, weeks, months, or years (e.g., at least seven days). In some embodiments, the model parameters may be continuously updated as more data is collected. Using the data from several days may increase robustness against noisy data. For example, in some embodiments, a first step in the model is to estimate the average bedtime and wakeup time whose variance decreases as more data is taken into account.

In some embodiments, in practice, the one or more sleep models may be built utilizing past sleep/wake information (as described above). Error! Reference source not found. shows an example of bedtime/wakeup time data used to estimate the parameters of a sleep model. In this model, days are used as the time unit.

TABLE 1
Example of bedtime/wakeup time data to estimate
the parameters of the base model

| | Bedtime | Wakeup time |
|---|---|---|
| Night index | [Day format] | [Day format] |
| 0 | 0.955 | 0.235 |
| 1 | 0.952 | 0.220 |
| 2 | 0.993 | 0.239 |
| 3 | 0.982 | 0.227 |
| 4 | 0.969 | 0.234 |
| 5 | 0.958 | 0.230 |
| 6 | 0.967 | 0.260 |
| 7 | 0.969 | 0.227 |

$$\begin{matrix}1 + ( {H}_{0} - 1 ) {e}^{- δ {W}_{i}} = {H}_{{e}^{′}} , & Equation 1 ( during wake ) \\ {H}_{e} {e}^{- σ {S}_{i}} = {H}_{0} , & Equation 2 ( during sleep ) \\ \begin{matrix}F ( {H}_{0} , σ , δ ) = {H}_{0} {e}^{σ {S}_{i}} + {e}^{- δ {W}_{i}} - \\ {H}_{0} {e}^{- δ {W}_{i}} \\ = 1\end{matrix} & Equation 3 \\ \frac{\partial F}{\partial {H}_{0}} = {e}^{σ {S}_{i}} - {e}^{- δ {W}_{i}} \\ \frac{\partial F}{\partial σ} = {S}_{i} {H}_{0} {e}^{σ {S}_{i}} \\ \frac{\partial F}{\partial δ} = - {W}_{i} {e}^{- δ {W}_{i}} + {H}_{0} {W}_{i} {e}^{- δ {W}_{i}} , & Equation 4 \\ {H}_{0} ← {H}_{0} - η \frac{\partial F}{\partial {H}_{0}} \\ σ ← σ - η \frac{\partial F}{\partial σ} \\ δ ← δ - η \frac{\partial F}{\partial δ} & Equation 5\end{matrix}$$

where Wᵢ and Sᵢ are the duration of wakefulness and sleep associated with day “i” respectively, and H₀ is the homeostatic threshold to transition from sleep to wake, Hₑ is the homeostatic threshold to transition from wake to sleep, τ_{w}=1/δ is the time constant controlling the accumulation of sleep-need during wake, and τₛ=1/σ is the time constant controlling the dissipation of sleep-need during sleep.
By matching Hₑ in Error! Reference source not found. and Error! Reference source not found., the function F to minimize in Error! Reference source not found. is obtained which has to be solved to estimate the values of H₀, σ, and δ. The iterative optimization Error! Reference source not found. (where) procedure uses the partial derivatives in Error! Reference source not found.

Returning to FIG. 1, in some embodiments, sleep prediction component 34 may be configured to predict one or more sleep parameters of the subject for an upcoming time interval. In some embodiments, the prediction may be based upon the sleep model. In some embodiments, the prediction may be based on the estimated bedtime and/or wakeup time. In some embodiments, the one or more sleep parameters may include sleep onset, sleep-need, sleep duration, and/or sleep parameters.

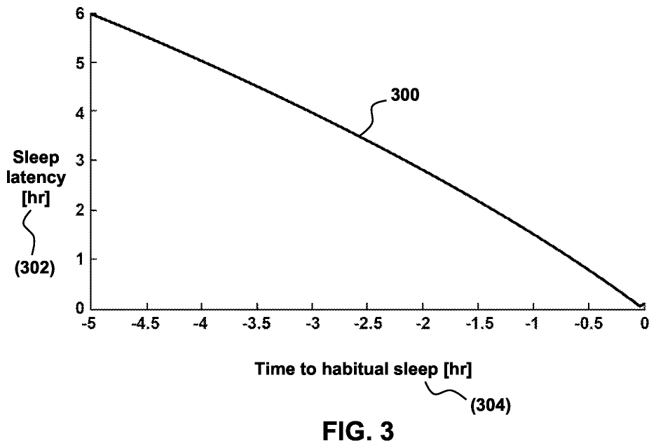

In some embodiments, the example of sleep model shown in FIG. 2 may be useful and practical to make predictions on sleep onset. For example, knowing the value of “H” for which wake transitions into sleep and the habitual bedtime, it is possible to determine sleep latency depending on the relative time difference with respect to habitual bedtime. FIG. 3 illustrates sleep latency prediction 300, in accordance with one or more embodiments. In FIG. 3, sleep latency prediction 300 depends on relative time difference 304 with respect to habitual bedtime. This prediction shows that as the habitual bedtime 304 approaches, sleep-need increases (sleep latency 302 decreases) at a slower pace which is reasonable given the exponentially saturating nature of the model during wake. For example, according to this prediction, going to bed an hour earlier may result in a sleep latency at least an hour longer compared to habitual sleep latency 302.

As described above, in some embodiments, one or more sensor signals and/or behaviors may be used to estimate H. These are not intended to limit the scope of this invention which covers the overall concept of utilizing daytime signals to adjust sleep predictions. In some embodiments, additional factors may be taken into consideration for a more accurate prediction. The sleep prediction may be adjusted (e.g. by sleep prediction component shown in FIG. 1). In some embodiments, these factors may include physiological information (and/or behavioral information) (e.g., obtained by physiological component 30, sleep component 32, and/or other components of system 10 as described above). In some embodiments, one of the factors may be physical activities information during wake time (as described above). For example, certain activities during wake may be more or less conducive to sleep (e.g. early exercise may promote faster sleep). Stress level, in some cases, may be one of the factors (e.g., high stress may increase sleep latency). Past sleep information may be another factor in some embodiments. For example, past history of wake/sleep which may have accumulated sleep-need may accelerate sleep latency (e.g., people who have accumulated “sleep debt” can go to bed earlier than usual and fall asleep faster than what is predicted).

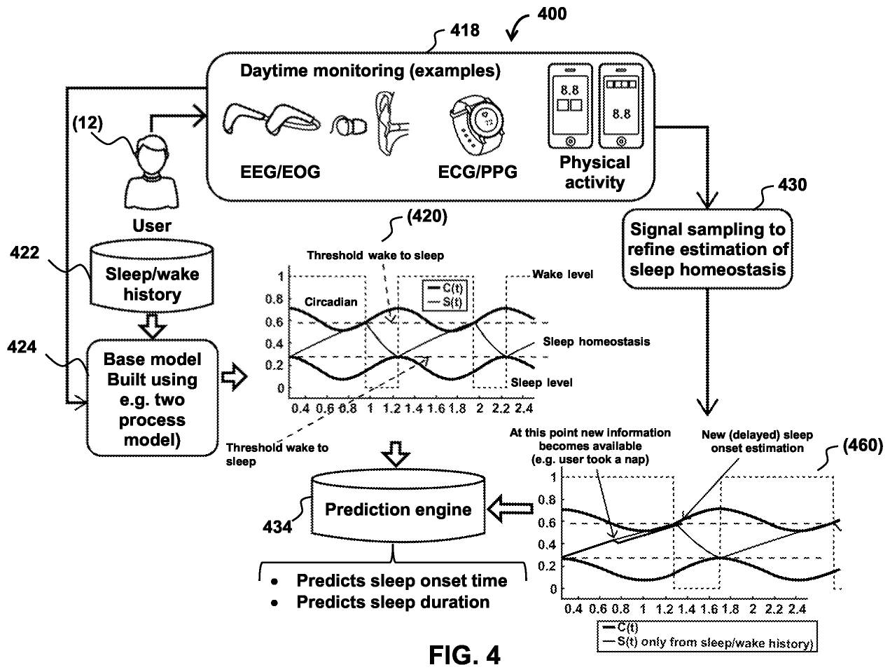

FIG. 4. illustrates an example operations 400 of system 10 according to one or more embodiments. It is to be understood that in some embodiments, system 10 does not strictly require monitoring of physiology and/or behavior of the subject. Instead, in some embodiments, it assumes the existence of monitoring means through third party devices (mobile phones, outside sensors, medical devices, computing devices, wearable devices, and/or other devices) which may interface with system 10. In some embodiments, sleep and/or wake information related to the subject for a given period of time is obtained from sleep/wake history component 422. Sleep/wake history component, in some embodiments, may include one or more databases (e.g., electronic storage 22, external sources 14, and/or other databases). In some embodiments, the sleep/wake history may be obtained from one or more devices 418 (described below).

In some embodiments, bedtime and/or wakeup time of the subject may be estimated using sleep model 424 and the sleep and/or wake information from sleep/wake history database 422. In some embodiments, sleep model 424 may be a two-process sleep model 420. In some embodiments, sleep model 420 is similar to sleep model 200 shown in FIG. 2 and described above. In some embodiments, one or more sleep parameters of the subject for an upcoming time interval may be predicted using prediction engine 434. In some embodiments, the one or more sleep parameters may be predicted based on sleep model 424 and the estimated bedtime and/or wakeup time. In some embodiments, the one or more sleep parameters may include sleep onset time, sleep-need, sleep debt, and/or sleep duration. In some embodiments, prediction engine 434 may be similar to sleep prediction component 34 shown in FIG. 1 and described above.

In some embodiments, physiological (and/or behavioral) information of the subject 12 may be obtained from one or more devices 418. In some embodiments, the physiological (and/or behavioral) information is similar to physiological information obtained by physiological parameter component 30 shown in FIG. 1 and described above. Devices 418 may (be, or) include one or more sensors (similar to sensors 18 shown in FIG. 1 and described above). As explained above, devices 418 (or sensors 18) may include one or more of a wearable device in the form of a wristband, an activity monitor, a smart watch, a headband, ear plugs, and/or wearable devices. In some embodiments, devices 418 (sensors 18) may include one or more computing devices (e.g., a mobile phone, a computer, an input device, etc.). In some embodiments, devices 418 (sensors 18) may include medical devices, and/or other monitoring devices.

In some embodiments, the predicted sleep parameters for the upcoming time interval may be adjusted based upon sleep model 420 and the physiological (and/or behavioral) information 430 obtained from devices 418. The adjusted model 460 shows that the homeostasis curve is adjusted, and a new delayed sleep onset estimation is determined in response to the new information (physiological and/or behavioral) 430 becoming available (e.g., the user took a nap). As explained above, at time “t” the signal s(t) is sampled and that a relation Sₕ exists such that an estimation of H at time t: [symbol] can be obtained from s(t): [symbol] =Sₕ(s(t)). The function Sₕ depends on the specific type of signal and specific instances thereof. The corrected value of H at time “t” that is utilized in model 460 to produce an adjusted estimation of sleep onset is: λ×H(t)+(1−λ)× [symbol] , where H(t) is the value originally predicted from the model and 0<λ<1 is a positive number that controls the degree of correction due to [symbol] .

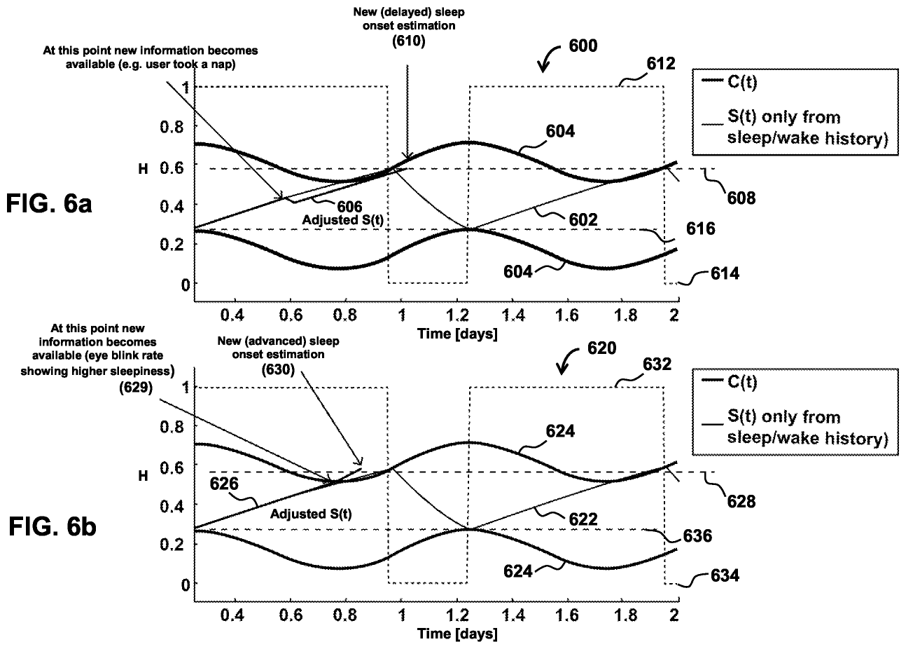

In some embodiments, the one or more physiological information obtained may include daytime sleep behavior information. In some embodiments, information about sleep behavior other than the night-time sleep of the user may be obtained. For example, in some cases, daytime napping is among behaviors that may influence night-time sleep. FIG. 6a illustrates an example of an adjusted sleep model 600 and an adjusted prediction based on the napping information. Sleep model 600 is a two-process model where a homeostatic component H models the accumulation of sleep-need during wakefulness 612 through an exponentially saturating curve (S(t))602. During sleep 614, the homeostatic component H models the dissipation of sleep-need using an exponentially decaying curve 602. In this model, the circadian factor determines the timing of sleep and wake through two “parallel” 24-hour period sinusoidal curves (C(t)) 604. A threshold wake to sleep 608 is defined by homeostatic saturating curve 602 meeting the circadian curve 604. A threshold sleep to wake 610 is defined by homeostatic decaying curve 602 meeting the circadian curve 604. However, at time “t” new physiological information 609 (the user took a nap) becomes available which causes adjustment 606 to homeostatic the saturating curve. An adjusted sleep onset 610 is estimated. The influence of napping at time “t” and for a duration of “T” days (this unit may be practical given the model framework) on the model prediction can be calculated by lowering the value of H(t+T) (i.e. H at time t+T) by:

$${\int }_{t}^{t + T} {H}_{e} exp ( \frac{{t}_{0} - t}{{τ}_{s}} ) dt .$$

As shown in FIG. 6a, since H decreases due to napping, sleep latency in subsequent night-time sleep increases.

In some embodiments, the one or more physiological information obtained may include daytime eye movement information. Eye movement and/or eyelid movement (e.g., blinks) are linked to vigilance, attention, and sleepiness. In some embodiments, one or more eye and/or lid movements may indicate a drowsiness level. For example, these one or more eye and/or lid movements may include percentage or duration of eye closure, blink duration, blink rate or blink amplitude, pendular motions, slow eye movements, lid closing/reopening time, interval between blinks, changes in pupil size, saccadic velocities, amplitude-velocity ratios of the eye closure, and/or other eye and/or lid movements. In some embodiments, daytime eye/lid movements of the subject may be monitored using sensors within and/or outside system 10. For example, the eye movement may be monitored using one or more sensors that incorporate detection and tracking of changes in the ocular region (e.g., ocular sensors, cameras, electrooculography (EOG), infrared reflectance oculography, etc.). In some embodiments, the eye/lid movement may be continually measured.

In some embodiments, physiological parameters component 30 may be configured to obtain/determine one or more daytime eye movement features based on the daytime eye movements. In some embodiments, the daytime eye movement features may be used to estimate a sleepiness scale. For example, the Karolinska Sleepiness Scale (KSS). KSS is a measure of a subjective level of sleepiness at a particular time during the day. The KSS is a 9-point Likert scale often used when conducting studies involving self-reported, subjective assessment of an individual's level of drowsiness at the time. On this scale, subjects indicate which level best reflects the psycho-physical state experienced in the last 10 min. The KSS Scores are defined as follows:
9. Extremely sleepy, fighting sleep
8. Sleepy, some effort to keep alert
7. Sleepy, but no difficulty remaining awake
6. Some signs of sleepiness
5. Neither alert nor sleepy
4. Rather alert
3. Alert
2. Very alert
1. Extremely alert

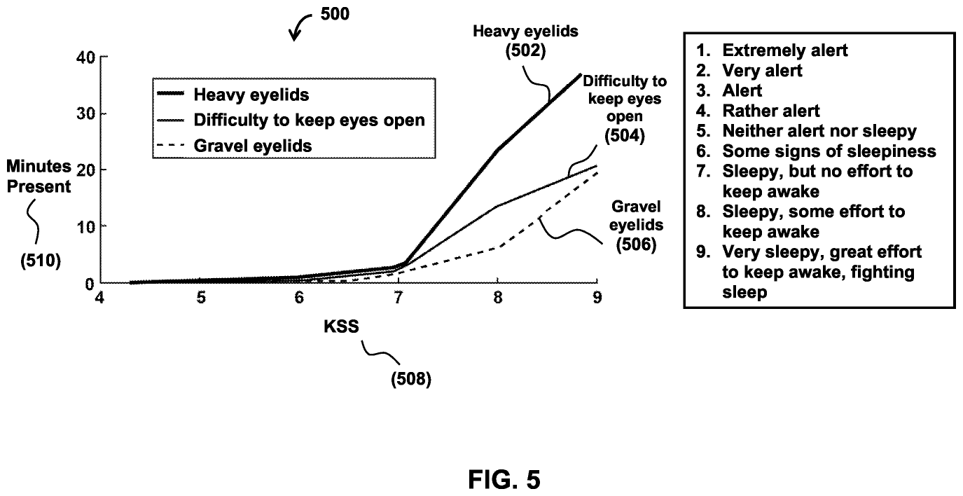

In general, KSS increases with longer periods of wakefulness and it correlates with the time of the day. FIG. 5 illustrates an example of the correlation between eye movement and sleepiness level according to one or more embodiments. The graphs in FIG. 5 show that eye region related features characterize sleepiness and are related to the scale KSS 508. For example, heavy eyelids graph 502 shows that the longer the subject experiences heavy eyelids, the sleepier he gets based on the scale KSS. The same is true for difficulty to keep eyes open 504 and gravel eyelids 506.

KSS from daytime eye movement derived features can be estimated as follows:

KSS≈1+ε×|iris−pupil|.

KSS can in turn be used to estimate H as follows:

KSS(t)≈9.68−0.46×1/H(t).

The type of adjustment on sleep onset prediction based on eye movement information is illustrated in FIG. 6b. FIG. 6b illustrates an adjusted model 620 and prediction based on updated information on alertness originating from eye-blink rates. Sleep model 620 is a two-process model where a homeostatic component H models the accumulation of sleep-need during wakefulness 632 through an exponentially saturating curve (S(t))622. During sleep 634, the homeostatic component H models the dissipation of sleep-need using an exponentially decaying curve 622. In this model, the circadian factor determines the timing of sleep and wake through two “parallel” 24-hour period sinusoidal curves (C(t)) 624. A threshold wake to sleep 628 is defined by homeostatic saturating curve 622 meeting the circadian curve 624. A threshold sleep to wake 636 is defined by homeostatic decaying curve 622 meeting the circadian curve 624. However, at time “t” new physiological information 629 (eye blink rate showing higher sleepiness) becomes available which causes adjustment 626 to the homeostatic saturating curve. An adjusted sleep onset 630 is estimated.

Other sleep parameters may be predicted using the sleep model. For example, in some embodiments, sleep duration may be predicted given bedtime “T_{b}” (measured from wakeup time). Indeed, the accumulated sleep-need up to time T_{b} is:

H(T_{b})=μ+(H₀−μ)exp((t₀−T_{b})/τ_{w}).

The duration of sleep is then calculated using the sleep-need dissipation formula:

Sleep duration=τₛ log(H(T_{b})/Hₛ),

where Hₛ is the threshold determining the transition from sleep to wake (as shown in FIG. 2).

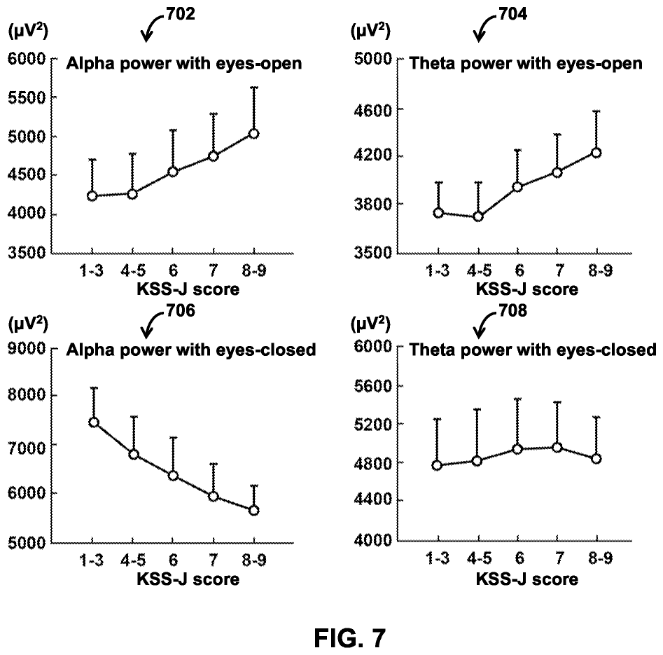

In some embodiments, the one or more physiological information may include daytime brain activity measurement (e.g., electroencephalography (EEG)). In some embodiments, changes in the subject's electroencephalography (EEG) measurements may indicate drowsiness in the subject. In some embodiments, EEG measurements may be obtained from one or more sensors within or outside system 10. In some embodiments, EEG based metrics include power in relevant frequency bands such as theta (4 to 8 Hz) and alpha (8-12 Hz). FIG. 7 illustrates a correlation between EEG metrics and scale KSS in accordance with one or more embodiments. Graph 702 shows the correlation between alpha power with eyes-open and the KSS. Graph 706 shows the correlation between alpha power with eyes-closed and the KSS. Graph 704 shows the correlation between theta power with eyes-open and the KSS. Graph 708 shows the correlation between theta power with eyes-closed and the KSS.

To estimate the corresponding values of H, the relation KSS(t)≈9.68−0.46×1/H(t) may be leveraged.

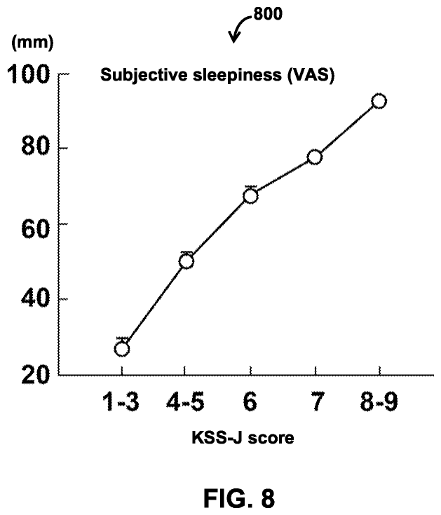

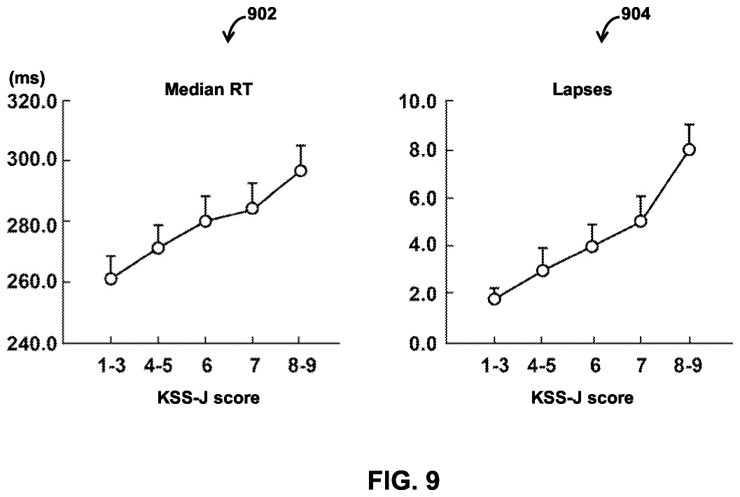

In some embodiments, the one or more physiological information may include subjective feedback on sleepiness obtained from the subject. For example, in some embodiments, under certain circumstances or due to professional requirements, it is possible to obtain subjective feedback on sleepiness typically using a visual analog scale (VAS). VAS correlates with KSS (see FIG. 8) and it can be used to estimate H. FIG. 8 illustrates a correlation 800 between subjective sleepiness as measured using the scale VAS and KSS. In some embodiments, performance on the psychomotor vigilance task (PVT) may indicate vigilance of the subject. PVT can generally be administered in two versions: 10-minutes or 3-minutes long. Because the latter is relatively short, it has been successfully used to quantify daytime vigilance. Significant correlations with KSS and two key PVT metrics (average reaction time and number of lapses) can be shown in FIG. 9. FIG. 9 illustrates examples of a correlation between PVT metrics (reaction time 902 and number of lapses 904).

In some embodiments, as shown in FIG. 1, system 10 may include one or more of external resources 14, electronic storage 22, client computing platform(s) 24, network 26, and/or other components, all being communicatively coupled via a network 26.

External resources 14 include sources of patient and/or other information. In some embodiments, external resources 14 include sources of patient and/or other information, such as databases, websites, etc., external entities participating with system 10 (e.g., a medical records system of a healthcare provider that stores medical history information for populations of patients), one or more servers outside of system 10, a network (e.g., the internet), electronic storage, equipment related to Wi-Fi technology, equipment related to Bluetooth® technology, data entry devices, sensors, scanners, and/or other resources. In some embodiments, some or all of the functionality attributed herein to external resources 14 may be provided by resources included in system 10. External resources 14 may be configured to communicate with processor 20, computing devices 24, electronic storage 22, and/or other components of system 10 via wired and/or wireless connections, via a network (e.g., a local area network and/or the internet), via cellular technology, via Wi-Fi technology, and/or via other resources.

Electronic storage 22 includes electronic storage media that electronically stores information. The electronic storage media of electronic storage 22 may include one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 22 may be (in whole or in part) a separate component within system 10, or electronic storage 22 may be provided (in whole or in part) integrally with one or more other components of system 10 (e.g., computing devices 24, processor 20, etc.). In some embodiments, electronic storage 22 may be located in a server together with processor 20, in a server that is part of external resources 14, in a computing device 24, and/or in other locations. Electronic storage 22 may include one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 22 may store software algorithms, information determined by processor 20, information received via a computing device 24 and/or graphical user interface 40 and/or other external computing systems, information received from external resources 14, sensors 18, and/or other information that enables system 10 to function as described herein.

Client computing platform(s) 24 is configured to provide an interface between system 10 and subject 12, and/or other users through which subject 12 and/or other users may provide information to and receive information from system 10. For example, client computing platform(s) 24 may display a representation of the output signal from sensors 18 (e.g., an EEG, 2D/3D images, video, audio, text, etc.) to a user. This enables data, cues, results, instructions, and/or any other communicable items, collectively referred to as “information,” to be communicated between a user (e.g., subject 12, a doctor, a caregiver, and/or other users) and one or more of processor 20, electronic storage 22, and/or other components of system 10.

Examples of interface devices suitable for inclusion in client computing platform(s) 24 comprise a keypad, buttons, switches, a keyboard, knobs, levers, a display screen, a touch screen, speakers, a microphone, an indicator light, an audible alarm, a printer, a tactile feedback device, and/or other interface devices. In some embodiments, client computing platform(s) 24 comprise a plurality of separate interfaces. In some embodiments, client computing platform(s) 24 comprise at least one interface that is provided integrally with processor 20, sensor(s) 18, and/or other components of system 10.

Computing devices 24 are configured to provide interfaces between caregivers (e.g., doctors, nurses, friends, family members, etc.), patients, and/or other users, and system 10. In some embodiments, individual computing devices 24 are, and/or are included, in desktop computers, laptop computers, tablet computers, smartphones, and/or other computing devices associated with individual caregivers, patients, and/or other users. In some embodiments, individual computing devices 24 are, and/or are included, in equipment used in hospitals, doctor's offices, and/or other medical facilities to patients; test equipment; equipment for treating patients; data entry equipment; and/or other devices. Computing devices 24 are configured to provide information to, and/or receive information from, the caregivers, patients, and/or other users. For example, computing devices 24 are configured to present a graphical user interface 40 to the caregivers to facilitate display representations of the data analysis and/or other information. In some embodiments, graphical user interface 40 includes a plurality of separate interfaces associated with computing devices 24, processor 20 and/or other components of system 10; multiple views and/or fields configured to convey information to and/or receive information from caregivers, patients, and/or other users; and/or other interfaces.

In some embodiments, computing devices 24 are configured to provide graphical user interface 40, processing capabilities, databases, and/or electronic storage to system 10. As such, computing devices 24 may include processors 20, electronic storage 22, external resources 14, and/or other components of system 10. In some embodiments, computing devices 24 are connected to a network (e.g., the internet). In some embodiments, computing devices 24 do not include processors 20, electronic storage 22, external resources 14, and/or other components of system 10, but instead communicate with these components via the network. The connection to the network may be wireless or wired. For example, processor 20 may be located in a remote server and may wirelessly cause display of graphical user interface 40 to the caregivers on computing devices 24. As described above, in some embodiments, an individual computing device 24 is a laptop, a personal computer, a smartphone, a tablet computer, and/or other computing devices. Examples of interface devices suitable for inclusion in an individual computing device 24 include a touch screen, a keypad, touch-sensitive and/or physical buttons, switches, a keyboard, knobs, levers, a display, speakers, a microphone, an indicator light, an audible alarm, a printer, and/or other interface devices. The present disclosure also contemplates that an individual computing device 24 includes a removable storage interface. In this example, information may be loaded into a computing device 24 from removable storage (e.g., a smart card, a flash drive, a removable disk, etc.) that enables the caregivers, patients, and/or other users to customize the implementation of computing devices 24. Other exemplary input devices and techniques adapted for use with computing devices 24 include, but are not limited to, an RS-232 port, an RF link, an IR link, a modem (telephone, cable, etc.), and/or other devices.

The network 26 may include the Internet and/or other networks, such as local area networks, cellular networks, Intranets, near field communication, frequency (RF) link, Bluetooth™, Wi-Fi™, and/or any type(s) of wired or wireless network(s). Such examples are not intended to be limiting, and the scope of this disclosure includes embodiments in which external resources 14, sensor(s) 18, processor(s) 20, electronic storage 22, and/or client computing platform(s) 24 are operatively linked via some other communication media.

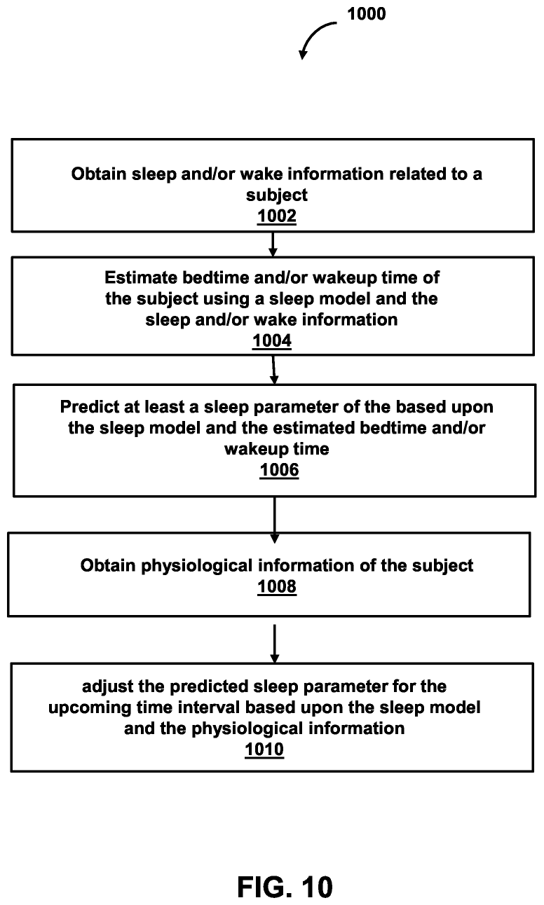

FIG. 10 illustrates a method 1000 for predicting at least a sleep parameter for a subject. The operations of method 1000 presented below are intended to be illustrative. In some embodiments, method 1000 may be accomplished with one or more additional operations not described, and/or without one or more of the operations discussed. Additionally, the order in which the operations of method 1000 are illustrated in FIG. 10 and described below is not intended to be limiting.

In some embodiments, method 1000 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 1000 in response to instructions stored electronically on an electronic storage medium. The one or more processing devices may include one or more devices configured through hardware, firmware, and/or software to be specifically designed for execution of one or more of the operations of method 1000.

At operation 1002, sleep and/or wake information related to a subject for a given period of time is obtained. In some embodiments, operation 1002 is performed by one or more processors the same as or similar to processors 20 (shown in FIG. 1 and described herein).

At operation 1004, bedtime and/or wakeup time of the subject is estimated using a sleep model and the sleep and/or wake information. In some embodiments, operation 1004 is performed by a physical computer processor the same as or similar to processor(s) 20 (shown in FIG. 1 and described herein).

At operation 1006, at least a sleep parameter of the subject for an upcoming time interval is predicted based upon the sleep model and the estimated bedtime and/or wakeup time. In some embodiments, operation 1006 is performed by a physical computer processor the same as or similar to processor(s) 20 (shown in FIG. 1 and described herein).

At operation 1008, physiological information of the subject is obtained. In some embodiments, operation 1008 is performed by a physical computer processor the same as or similar to processor(s) 20 (shown in FIG. 1 and described herein).

At operation 1010, the predicted sleep parameter for the upcoming time interval is predicted based upon the sleep model and the physiological information. In some embodiments, operation 1010 is performed by a physical computer processor the same as or similar to processor(s) 20 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

What is claimed is:

1. A non-transitory computer readable medium having instructions thereon, the instructions when executed by a computer causing the computer to:
obtain sleep and/or wake information related to a subject for a given period of time;
estimate bedtime and/or wakeup time of the subject using a sleep model and the sleep and/or wake information;
predict at least a sleep parameter of the subject for an upcoming time interval based upon the sleep model and the estimated bedtime and/or wakeup time;
obtain physiological information of the subject; and
adjust the predicted sleep parameter for the upcoming time interval based upon the sleep model and the physiological information.

2. The non-transitory computer readable medium of claim 1, further configured to obtain an average sleep and/or wake time over the given period.

3. The non-transitory computer readable medium of claim 1, wherein the sleep parameters comprise one or more of a sleep duration, sleep onset, and/or sleep-need.

4. The non-transitory computer readable medium of claim 1, wherein the physiological information comprises one or more behavior information of the subject, the one or more behavior affecting the sleep parameter of the subject.

5. The non-transitory computer readable medium of claim 1, wherein the sleep model is a two-process model comprising a homeostatic and a circadian component.

6. The non-transitory computer readable medium of claim 5, wherein the homeostatic component of the sleep model is updated based on the obtained physiological information.

7. A method for modeling at least a sleep parameter for a subject, the method comprising:
obtaining, with one or more processors, sleep and/or wake information related to a subject for a given period of time;
estimating, with one or more processors, bedtime and/or wakeup time of the subject using a sleep model and the sleep and/or wake information;
predicting, with one or more processors, at least a sleep parameter of the subject for an upcoming time interval based upon the sleep model and the estimated bedtime and/or wakeup time;
obtaining, with one or more processors, physiological information of the subject; and
adjusting, with one or more processors, the predicted sleep parameter for the upcoming time interval based upon the sleep model and the physiological information.

8. The method of claim 7, further comprising obtaining an average sleep and/or wake time over the given period

9. The method of claim 7, wherein the sleep parameters comprise one or more of a sleep duration, sleep onset, and/or sleep-need.

10. The method of claim 7, wherein the physiological information comprises one or more behavior information of the subject, the one or more behavior affecting the sleep parameter of the subject.

11. The method of claim 7, wherein the sleep model is a two-process model comprising a homeostatic and a circadian component.

12. The method of claim 11, wherein the homeostatic component of the sleep model is updated based on the obtained physiological information.

13. A system for modeling at least a sleep parameter for a subject, the system comprising:
obtaining means for obtaining sleep and/or wake information related to a subject for a given period of time;
estimating means for estimating bedtime and/or wakeup time of the subject using a sleep model and the sleep and/or wake information;
predicting means for predicting at least a sleep parameter of the subject for an upcoming time interval based upon the sleep model and the estimated bedtime and/or wakeup time;
obtaining means for obtaining physiological information of the subject; and
adjusting means for adjusting the predicted sleep parameter for the upcoming time interval based upon the sleep model and the physiological information.

14. The system of claim 13, further comprising obtaining an average sleep and/or wake time over the given period.

15. The system of claim 13, wherein the sleep parameters comprise one or more of a sleep duration, sleep onset, and/or sleep-need.

16. The system of claim 13, wherein the physiological information comprises one or more behavior information of the subject, the one or more behavior affecting the sleep parameter of the subject.

17. The system of claim 16, wherein the sleep model is a two-process model comprising a homeostatic and a circadian component.

18. The system of claim 13, wherein the homeostatic component of the sleep model is updated based on the obtained physiological information.

19. A system for modeling at least a sleep parameter for a subject, the system comprising:
one or more input devices configured to generate output signals indicating one or more physiological information related to a subject; and
one or more physical processors operatively connected with the one or more input devices, the one or more physical processors configured by machine-readable instructions to:
obtain sleep and/or wake information related to a subject for a given period of time;
estimate bedtime and/or wakeup time of the subject using a sleep model and the sleep and/or wake information;
predict at least a sleep parameter of the subject for an upcoming time interval based upon the sleep model and the estimated bedtime and/or wakeup time;
obtain physiological information of the subject; and
adjust the predicted sleep parameter for the upcoming time interval based upon the sleep model and the physiological information.

20. A non-transitory computer readable medium having instructions thereon, the instructions when executed by a computer causing the computer to:
obtain sleep and/or wake information related to a subject for a given period of time;
estimate bedtime and/or wakeup time of the subject using a sleep model and the sleep and/or wake information, the sleep model having one or more model parameters;
predict one or more sleep parameters, the one or more sleep parameters including sleep onset of the subject for an upcoming time interval based upon the sleep model and the estimated bedtime and/or wakeup time, wherein predicting the sleep onset includes predicting timing of a first sleep model parameter reaching a first upper sleep model parameter threshold;
obtain physiological information of the subject, the physiological information including a first physiological information of the subject; and
adjust the predicted sleep onset based upon the sleep model and the first physiological information, wherein adjusting the predicted sleep onset comprises adjusting the first sleep model parameter based on the first physiological information, wherein the first physiological information causes a decrease in the first sleep model parameter.

21. The non-transitory computer readable medium of claim 20, wherein the instructions further cause the computer to:
adjust the predicted sleep onset based upon the sleep model and a second physiological information, wherein adjusting the predicted sleep onset comprises adjusting the first sleep model parameter based on the second physiological information, wherein the second physiological information causes an increase in the first sleep model parameter.

22. The non-transitory computer readable medium of claim 20, wherein:
the one or more model parameters comprise homeostatic pressure;
predicting the sleep onset includes predicting a first timing of the homeostatic pressure reaching a first homeostatic pressure threshold indicating a change from a wake status to a sleep status of the subject;
the first physiological information comprises daytime sleep information; and
adjusting the predicted sleep onset comprises adjusting the homeostatic pressure based on the daytime sleep information, such that the daytime sleep information causes a decrease in the homeostatic pressure, and wherein adjusting the predicted sleep onset includes predicting a second timing of the homeostatic pressure reaching the first homeostatic pressure threshold.

23. The non-transitory computer readable medium of claim 22, wherein the second timing of the homeostatic pressure reaching the first homeostatic pressure threshold is later than the first timing.

24. The non-transitory computer readable medium of claim 21, wherein:
the one or more model parameters comprise homeostatic pressure;
predicting the sleep onset includes predicting a first timing of the homeostatic pressure reaching a first homeostatic pressure threshold indicating a change from a wake status to a sleep status of the subject;
the second physiological information comprises eye movement information; and
adjusting the predicted sleep onset comprises adjusting the homeostatic pressure based on the eye movement information, such that the eye movement information causes an increase in the homeostatic pressure, and wherein adjusting the predicted sleep onset includes predicting a third timing of the homeostatic pressure reaching the first homeostatic pressure threshold.

25. The non-transitory computer readable medium of claim 24, wherein the third timing of the homeostatic pressure reaching the first homeostatic pressure threshold is earlier than the first timing.
